(19) United States
(12) Patent Application Publication
(10) Pub. No.: US 2026/0324842 A1
(43) Pub. Date: Oct. 22, 2026

(54) WEARABLE STORAGE CONTAINER FOR PLASTIC GLOVES

(71) Applicant: Megan Arender, Lead Hill, AR (US)

(72) Inventor: Megan Arender, Lead Hill, AR (US)

(21) Appl. No.: 19/180,970

(22) Filed: Apr. 16, 2025

(51) Int. Cl. A45F 5/06; A45F 5/02; A61B 42/40; B65D 83/08

(52) U.S. Cl. CPC A45F 5/06; A45F 5/021; A61B 42/40; B65D 83/0805

(57) ABSTRACT

A wearable storage container for plastic gloves includes a container, a clip, a plurality of plastic gloves, and a pocket. The container includes an orifice connected to an interior compartment. The clip is coupled to the container. The clip is designed to slide onto and be suspended from an article of clothing. The plastic gloves are positioned in the interior compartment. The plastic gloves are removable one at a time through the orifice. The pocket is positioned on the container. The pocket is designed to hold personal accessories and equipment.

### B. CROSS-REFERENCE TO RELATED APPLICATIONS

Not Applicable

### C. STATEMENT REGARDING FEDERALLY SPONSORED RESEARCH OR DEVELOPMENT

Not Applicable

### D. THE NAMES OF THE PARTIES TO A JOINT RESEARCH AGREEMENT

Not Applicable

### E. INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### F. STATEMENT REGARDING PRIOR DISCLOSURES BY THE INVENTOR OR JOINT INVENTOR

Not Applicable

### G. BACKGROUND OF THE INVENTION

### 1. Field of the Invention

The disclosure relates to wearable storage devices and more particularly pertains to a new wearable storage container for holding a supply of plastic gloves, such as disposable nitrile gloves used in the medical fields and other industries. Most containers of disposable gloves are located in a fixed location or box placed in a room. Accordingly, a person seeking to wear such gloves must walk to the location or there must be multiple boxes placed in many areas in a building, such as a hospital or medical center. The new wearable storage container is designed to be worn around so that the gloves are immediately accessible at all times.

### 2. Description of Related Art including information disclosed under 37 CFR 1.97 and 1.98

The prior art relates to wearable storage devices. The prior art, as best understood, does not disclose a wearable storage container for plastic gloves that includes a container, a clip, a plurality of plastic gloves, and a pocket, wherein the clip is designed to slide onto and be suspended from an article of clothing, and the pocket is designed to hold personal accessories and equipment.

### H. BRIEF SUMMARY OF THE INVENTION

An embodiment of the disclosure meets the needs presented above in a wearable storage container generally comprising a container, a clip, a plurality of plastic gloves, and a pocket. The container includes an orifice connected to an interior compartment. The clip is coupled to the container. The clip is designed to slide onto and be suspended from an article of clothing. The plastic gloves are positioned in the interior compartment. The plastic gloves are removable one at a time through the orifice. The pocket is positioned on the container. The pocket is designed to hold personal accessories and equipment.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### I. BRIEF DESCRIPTION OF SEVERAL VIEWS OF THE DRAWINGS

The disclosure will be better understood and objects other than those set forth above will become apparent when consideration is given to the following detailed description thereof. Such description makes reference to the annexed drawings wherein:

FIG. 1 is a top rear perspective view of a wearable storage container according to an embodiment of the disclosure.

FIG. 2 is a top front perspective view of an embodiment of the disclosure.

FIG. 3 is a top view of an embodiment of the disclosure.

FIG. 4 is a side view of an embodiment of the disclosure in use.

FIG. 5 is a side view of an embodiment of the disclosure in use.

### J. DETAILED DESCRIPTION OF THE INVENTION

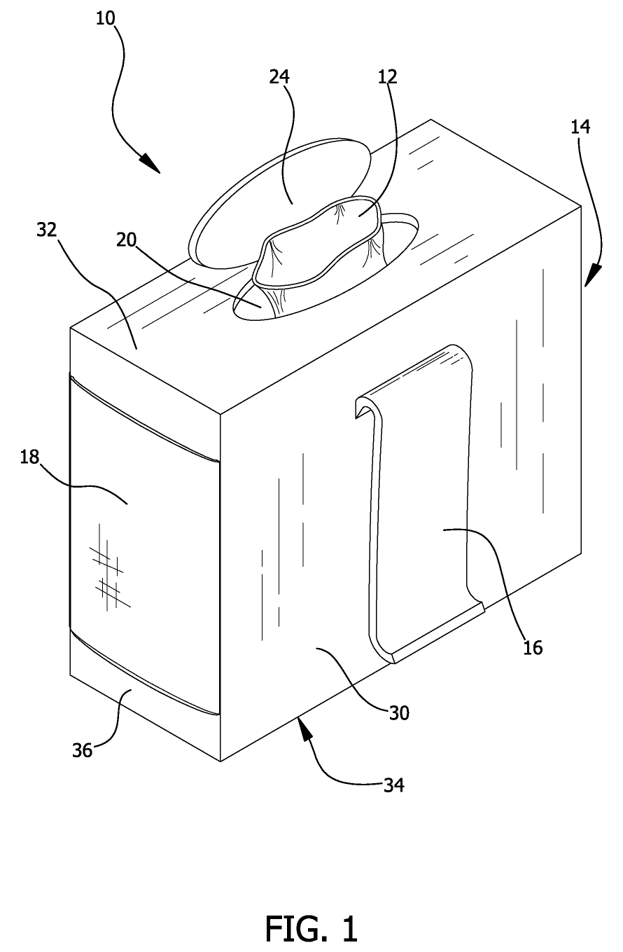

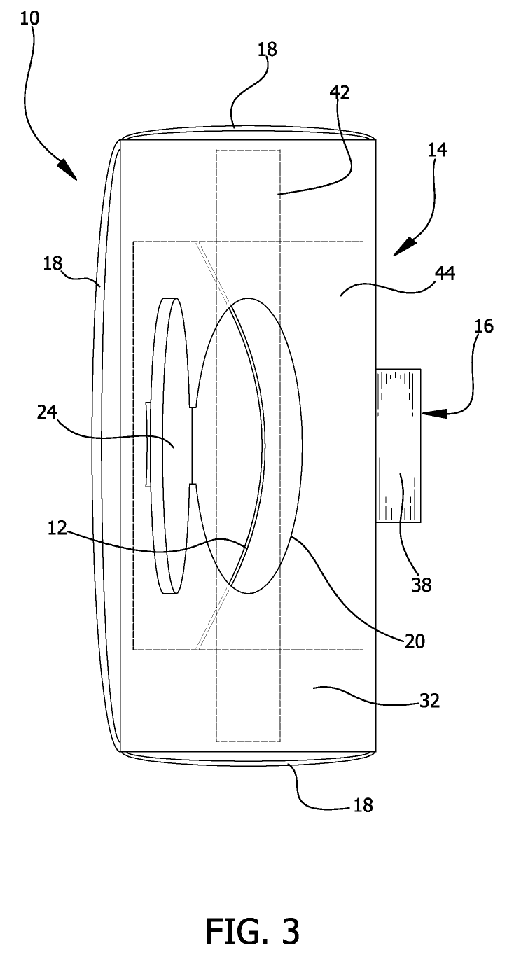

With reference now to the drawings, and in particular to FIGS. 1-5 thereof, a new wearable storage container embodying the principles and concepts of an embodiment of the disclosure and generally designated by the reference numeral 10 will be described.

As best illustrated in FIGS. 1-5, the wearable storage container 10 for plastic gloves 12 generally comprises a container 14, a clip 16, a plurality of plastic gloves 12, and a pocket 18. The container 14 includes an orifice 20 connected to an interior compartment 22. The clip 16 is coupled to the container 14. The clip 16 is designed to slide onto and be suspended from an article of clothing, such as a belt, waistband, or visor, for example. The plastic gloves 12 are positioned in the interior compartment 22. The plastic gloves 12 are removable one at a time through the orifice 20. The pocket 18 is positioned on the container 14. The pocket 18 is designed to hold personal accessories and equipment or other small items.

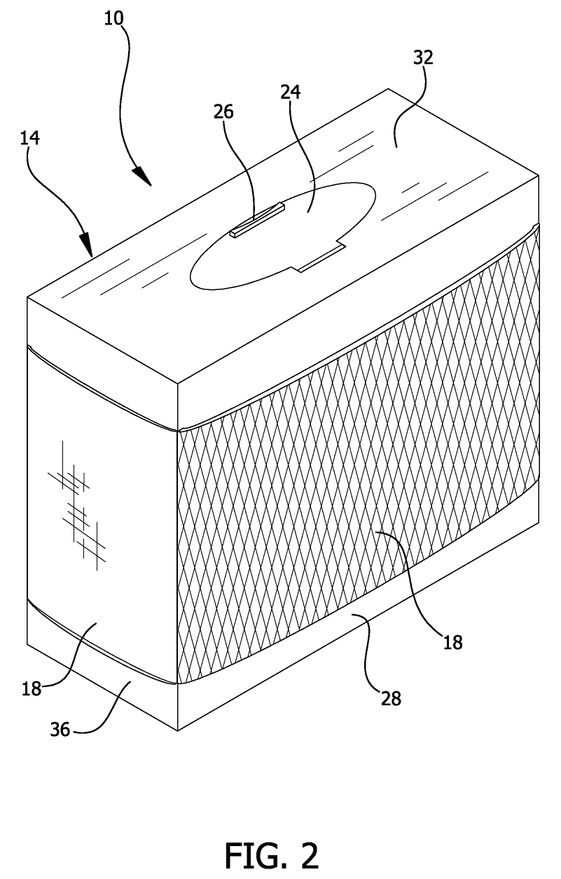

The wearable storage container 10 further includes a lid 24. The lid 24 is pivotably attached to the container 14 to selectively open and close the orifice 20. In the exemplary embodiment in FIG. 1, the lid 24 and the orifice 20 have an oval shape, though other shapes such as circular and square are within the scope of the disclosure. As seen in FIG. 2, the lid 24 is closed over the orifice 20 and includes a handle 26 for grasping the lid 24.

The container 14 is box-shaped and includes a front wall 28, a back wall 30, a top wall 32, a bottom wall 34, and two side walls 36. The container 14 could also be semi-circular or rounded. In one possible embodiment, the container 14 is made of rigid or semi-rigid plastic or similar material.

In the exemplary embodiment in FIG. 1, the lid 24 is attached to the top wall 32. The orifice 20 is positioned in the top wall 32.

In the exemplary embodiment in FIG. 1, the clip 16 is attached to the back wall 30 adjacent the top wall 32. The clip 16 extends along the back wall 30 from the top wall 32 toward the bottom wall 34. The clip 16 has a standard design common to belt and visor clips, with a base portion 38 attached to the bottom wall 34 and an extension portion 40 extending from the base portion 38 at an inward angle. In another possible embodiment, a different structure than the clip 16 could be used, such as snaps, magnets, straps, or hook and loop strips.

The pocket 18 is positioned on one of the front wall 28 and the side walls 36. The pocket 18 can include a layer of resiliently stretchable material, such as a stretchable fabric or cloth or elastic material, such as a mesh as shown in FIG. 2. In the exemplary embodiment in the figures, there are three pockets 18, wherein each of the pockets 18 is positioned on a respective one of the front wall 28 and the side walls 36. In another possible embodiment, the three pockets 18 are formed from a single band of resilient material attached at the back wall 30 and extending around the side walls 36 and the front wall 28.

The plastic gloves 12 are nitrile gloves, though other plastic gloves 12, such as latex gloves, could be in the container 14.

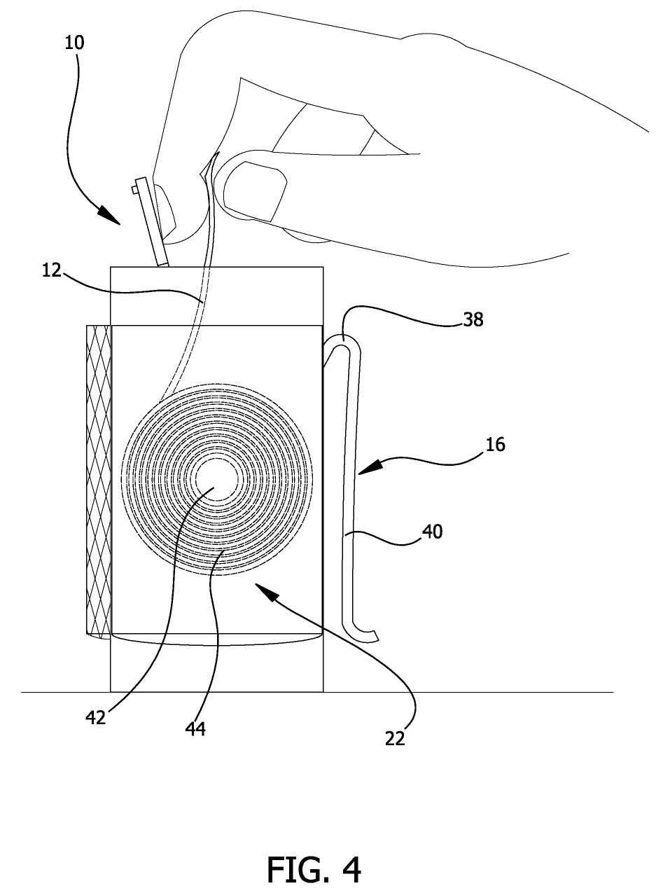

The wearable storage container 10 further includes a tube 42 positioned inside the container 14. The gloves 12 are in a roll 44 on the tube 42 and removable one at a time when the roll 44 is unrolled, as shown in FIG. 4.

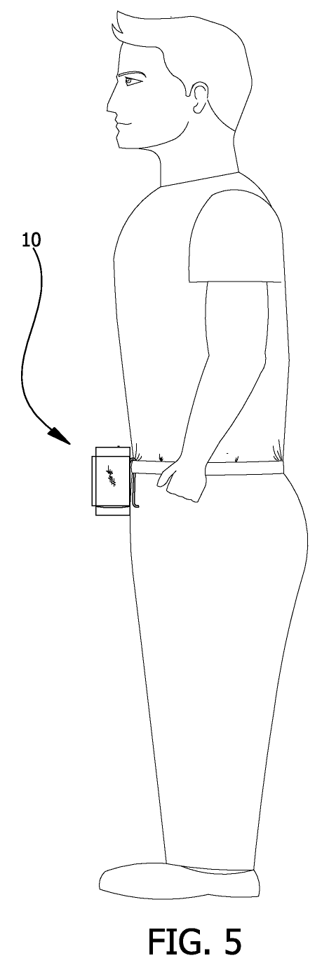

As shown in FIG. 5, the wearable storage container 10 can be clipped onto an article of clothing, such as a belt. Personal items or work accessories can also be stored in the pocket 18, such as keys, a badge, or a mobile phone. The user has easy access to the gloves 12 at all times while wearing the wearable storage container 10.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A wearable storage container for plastic gloves comprising:
a container, the container including an orifice connected to an interior compartment;
a clip, the clip being coupled to the container, the clip being configured to slide onto and be suspended from an article of clothing;
a plurality of plastic gloves, the plastic gloves being positioned in the interior compartment, the plastic gloves being removable one at a time through the orifice; and
a pocket, the pocket being positioned on the container, the pocket being configured to hold personal accessories and equipment.

2. The wearable storage container of claim 1, wherein the container is box-shaped and includes a front wall, a back wall, a top wall, a bottom wall, and two side walls.

3. The wearable storage container of claim 2, further comprising a lid, the lid being pivotably attached to the container to selectively open and close the orifice.

4. The wearable storage container of claim 3, wherein:
the lid is attached to the top wall; and
the orifice is positioned in the top wall.

5. The wearable storage container of claim 2, wherein:
the clip is attached to the back wall adjacent the top wall; and
the clip extends along the back wall from the top wall toward the bottom wall.

6. The wearable storage container of claim 2, wherein the pocket is positioned on one of the front wall and the side walls.

7. The wearable storage container of claim 6, further comprising two additional pockets, each of the pockets being positioned on a respective one of the front wall and the side walls.

8. The wearable storage container of claim 1, wherein the pocket comprises a layer of resiliently stretchable material.

9. The wearable storage container of claim 1, wherein the plastic gloves are nitrile gloves.

10. The wearable storage container of claim 1, further including a tube positioned inside the container, wherein the gloves are in a roll on the tube and removable one at a time when the roll is unrolled.

11. The wearable storage container of claim 4, wherein:
the clip is attached to the back wall adjacent the top wall; and
the clip extends along the back wall from the top wall toward the bottom wall.

12. The wearable storage container of claim 11, wherein the pocket is positioned on one of the front wall and the side walls.

13. The wearable storage container of claim 12, further comprising two additional pockets, each of the pockets being positioned on a respective one of the front wall and the side walls.

14. The wearable storage container of claim 13, wherein the pocket comprises a layer of resiliently stretchable material.

15. The wearable storage container of claim 14, wherein the plastic gloves are nitrile gloves.

16. The wearable storage container of claim 15, further including a tube positioned inside the container, wherein the gloves are in a roll on the tube and removable one at a time when the roll is unrolled.
